(19) United States
(12) Patent Application Publication
(10) Pub. No.: US 2009/0248081 A1
(43) Pub. Date: Oct. 1, 2009

(54) Spinal Stabilization Devices and Methods

(75) Inventors: Jean Charles LeHuec, Pessac (FR); Hallett H. Mathews, Williamsburg, VA (US); Mingyan Liu, Bourg-la-Reine (FR)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/059,501

(22) Filed: Mar. 31, 2008

(51) Int. Cl. A61B 17/70

(52) U.S. Cl. 606/263; 606/246

(57) ABSTRACT

A device and method for stabilizing a spine utilizes one or more stabilization members made of, or including, a bioresorbable and/or biointegrable material such as natural tissue or a bioresorbable polymer. The stabilization member(s) may be elastic, and may secure one or more spinal motion segments in a manner effective to reduce the range of flexion and/or extension of the spinal motion segments. The stabilization member may include one or more elongate straps of nonosteogenic natural tissue, each of which may be secured to the spine using fasteners such as bone screws or tacks. The stabilization device may include a blocking member sized and configured to be effective for maintaining a medically desirable distance between adjacent spinous processes in the spine of a medical patient.

### FIELD OF THE INVENTION

The present invention relates generally to devices and methods for stabilizing a spine, and more particularly to devices and methods for stabilizing a spine against excessive extension or compression.

### BACKGROUND OF THE INVENTION

Intervertebral stabilizers are known to be used to prevent excessive flexion or extension motion of the spinal column. The most aggressive stabilizers are metal plates or rods that are fixed to the vertebrae along the affected region to completely immobilize the vertebrae. Such stabilizers have the drawback of severely restricting, or even preventing, flexional and extensional movement, thus reducing patient mobility. In addition, rigid metal constructs may intrude into the adjacent tissue and vasculature, and may require multiple surgeries to install and maintain.

More recently, flexible stabilizers have been developed to overcome disadvantages of the prior art metal stabilizers. For example, U.S. Pat. No. 6,652,585 to Lange discloses a spine stabilization system that includes a flexible member attachable to a portion of the spinal column. The member includes components that are oriented and function similar to the natural fiber orientation of the anterior longitudinal ligament and annulus tissue, to resist extensional and rotational loading while maintaining some degree of flexibility. U.S. Pat. No. 6,852,128, also to Lange, discloses other flexible spine stabilization systems with similar characteristics.

The components of the prior art flexible stabilization systems are made from one or a combination of metal materials, polymeric materials, ceramic materials, shape memory materials, and composites thereof. While those materials may provide advantages over rigid metal stabilizers, they may fail to provide either a satisfactory mechanical life or optimal biomechanical performance.

In addition to controlling excessive flexion or extension motion of the spinal column, devices for controlling excessive compression are also known. These devices are particularly useful for treating patients suffering from lumbar spinal stenosis (“LSS”, and sometimes called sciatica), a condition of the spine characterized by a narrowing of the lumbar spinal canal. With spinal stenosis, the spinal canal narrows and pinches the spinal cord and nerves, causing pain in the back and legs. It is estimated that approximately 5 in 10,000 people develop LSS each year. For patients who seek the aid of a physician specialist for back pain, approximately 12-15% are diagnosed as having LSS.

Several causes of spinal stenosis have been identified, including aging, heredity, arthritis, and changes in blood flow to the lower spine. Aging is believed to be the most common cause, because as a person ages the ligaments connecting the bones of the spine can thicken and spurs may develop on the bones and into the spinal canal. The cushioning discs between the vertebrae also frequently deteriorate, and the facet joints may begin to break down. Heredity is believed to play a role in some cases because it may cause some people to have a smaller than average spinal canal, typically leading to LSS symptoms even at a relatively young age.

The most common symptom of spinal stenosis is pain and difficulty when walking, although numbness, tingling, hot or cold feelings in the legs, and weakness or tiredness may also be experienced. In extreme cases spinal stenosis can cause cauda equina syndrome, a syndrome characterized by neuromuscular dysfunction that may result in permanent nerve damage.

Common treatments for LSS include physical therapy (including changes in posture), medication, and occasionally surgery. Changes in posture and physical therapy may be effective in flexing the spine to enlarge the space available to the spinal cord and nerves—thus relieving pressure on pinched nerves. Medications such as NSAIDS and other anti-inflammatory medications are often used to alleviate pain, although they are not typically effective at addressing the cause of the pain. Surgical treatments are more aggressive than medication or physical therapy, but in appropriate cases, surgery may be the best way to achieve a lessening of the symptoms associated with LSS.

The most common surgery for treating LSS is decompressive laminectomy, in which the lamina of one or more vertebrae is removed to create more space for the nerves. The intervertebral disc may also be removed, and the vertebrae may be fused to strengthen unstable segments. The success rate of decompressive laminectomy has been reported to be in excess of 65%, with a significant reduction in LSS symptoms being achieved in many cases.

More recently, a second surgical technique has been developed in which the vertebrae are distracted and an interspinous process spacer is implanted to maintain the desired separation between the segments. This technique is somewhat less invasive than decompressive laminectomy, but may provide significant benefits to patients experiencing LSS symptoms.

Prior art interspinous spacers have been made of synthetic materials that provide long implant life and satisfactory spacing and cushioning capability. While such spacers have found utility when properly made and used, they may not provide the mechanical and immunological properties that are most desired.

A need therefore exists for intervertebral stabilizers that protect against excessive flexion and extension of spinal motions segments, while still allowing optimal biomechanical performance. A need also exists for interspinous spacers that have improved mechanical and immunological properties. The present invention addresses at least one of these needs.

### SUMMARY OF THE INVENTION

In one aspect of the present invention there is provided a spinal stabilization device made of a bioresorbable and/or biointegrable material such as natural tissue. The device for stabilizing a spinal motion segment includes one or more stabilizing members, such as straps or bands, made of a nonosteogenic bioresorbable material effective for stabilizing a spine against excessive flexion or extension. The strap(s) may be secured to the spine with fasteners such as bone screws or tacks.

The device may additionally or alternatively include a nonosteogenic bioresorbable blocking member sized and configured to be effective for stabilizing a spine against excessive compression. The blocking member is effective for maintaining a medically desired distance between two, adjacent spinous processes when the natural tissue blocking member is positioned between the two spinous processes in the spine of a human patient.

### BRIEF DESCRIPTION OF THE DRAWINGS

FIGS. 1A-1C show stabilization devices according to one preferred embodiment of the present invention.

FIGS. 2A-2D show the stabilization devices of FIGS. 1A-1C implanted to stabilize a spine.

FIGS. 3A and 3B show another view of the stabilization device of FIG. 1A implanted to stabilize a spine.

FIG. 4 shows a stabilization device including a blocking member according to another embodiment of the present invention.

FIG. 5 shows a stabilization device including a blocking member according to another embodiment of the present invention.

FIGS. 6A-6B show a stabilization device including a blocking member implanted to stabilize a spine.

### DETAILED DESCRIPTION

For the purposes of promoting an understanding of the principles of the invention, reference will now be made to certain embodiments thereof and specific language will be used to describe the same. It will nevertheless be understood that no limitation of the scope of the invention is thereby intended, such alterations and further modifications of the disclosed embodiments being contemplated as would normally occur to one skilled in the art to which the invention relates.

As indicated above, one aspect of the present invention relates to stabilizers for stabilizing a spine, wherein the stabilizers comprise, consist of, or consist essentially of a bioresorbable, nonosteogenic, and/or biointegrable material such as natural tissue. In some embodiments the stabilizers may be used to stabilize a single vertebra, and thus to protect against excessive and/or undesired motion of that segment. In other embodiments the stabilizers may be used to hold two to more spinal motion segments together or apart, and thus to restrict or control the range of motion, and particularly separation, of the joined segments. The stabilizers, or stabilization members, may be nonosteogenic, bioresorbable, and/or biointegrable.

In one embodiment the stabilizer comprises an elongate strap of nonosteogenic natural tissue securable to a pair of spinal motion segments. The strap is sized and configured to prevent the spinal motion segments from moving in undesired directions, and typically to prevent excessive flexion and/or extension, when both ends of the strap are secured. A first end of the stabilizing strap may be secured to a first spinal motion segment using a means for securing, such as bioacceptable screws, nails, staples, etc. A second end of the stabilizing strap may be secured to a second spinal motion segment using the same or different method of securement. When both ends of the stabilizer are secured, the two spinal motion segments are stabilized against excessive, undesired motion, such as excessive separation between the two segments.

When natural tissue is used to make the stabilizer of the present invention, the natural tissue may be derived from substantially any natural tissue providing the appropriate mechanical and immunological properties, i.e., any natural tissue capable of providing the correct compliance and implant substance to be integrated into the body and allow for stabilizing spinal motion segments. In some embodiments the natural tissue may comprise and/or be derived from soft tissue such as skin, pericardium, tendon, ligament, fascia, muscle, cartilage, intestine, meniscus, etc. Examples of preferred sources of natural tissue include fascia lata, planar fascia, anterior or posterior cruciate ligaments, patella tendon, hamstring tendons, quadriceps tendons, Achilles tendons, skins, and other connective tissues. The material may be autogenic (autograft), allogenic (allograft), or xenogenic (xenograft), or it may be of human-recombinant origin. The tissue can be of human origin (e.g. for allograft or autograft into human patients) or of non-human origin (e.g. for xenograft implant into human patients).

In some embodiments the biorespobable material is a nonosteogenic natural tissue. Examples of such nonosteogenic natural tissue include fascia lata, planar fascia, anterior or posterior cruciate ligaments, patella tendon, hamstring tendons, quadriceps tendons, Achilles tendons, skins, and other connective tissues that have not been treated to make them osteogenic.

The tissue used in certain embodiments of the present invention may be treated with a cross-linking agent to promote cross-linking of the collagen molecules in the tissue. For example, glutaraldehyde or other protein cross-linking agents may be used to promote covalent or non-covalent crosslinks between collagen molecules. Similarly, agents to inhibit protein denaturation may also be included. Crosslinking agents that would be appropriate for use in the claimed invention are known to persons skilled in the art, and may be selected without undue experimentation.

When natural tissue is used, the tissue may be used in its fresh form, or it may be frozen and/or dehydrated before use. In some embodiments the natural tissue material is tissue that has not been processed other than to affect its size or shape.

In other embodiments a bioresorbable and/or biointegrable material other than natural tissue may be used. For example, all or portions of the inventive stabilizers may comprise one or more bioresorbable polymers such as poly-L-lactic acid (PLLA), poly-DL-lactic acid (PDLLA), and/or polylactide (PLa, a copolymer of 70:30 Poly(L-lactide-co-D,L-lactide). In some embodiments the synthetic bioresorbable material is nonosteogenic.

It is to be appreciated that in some embodiments at least a portion of the inventive stabilizers may be made from materials that are not bioresorbable, including natural or synthetic polymers, rubbers, metals, ceramics, and/or composites.

Regardless of the material used, the stabilizers may have elastic properties so that initially they stretch when subject to movement of the spine, yet they resist further stretching when excessive movement might otherwise occur. The stabilizers thus may provide elastic forces to return the spine to its proper alignment before excessive movement, such as excessive flexion, extension, or compression, can occur.

The amount of motion or separation of spinal motion segments that is “excessive” may vary from case to case. Typically, medical personnel will identify a desired maximum range of motion for a spinal region or segment in a patient, and will thus identify the amount of flexion and/or extension and/or compression that is deemed to be excessive for that/those spinal segment(s).

The stabilizers of the present invention may be connected, attached, secured, fitted, etc., (collectively, connected) to a variety of locations on a spinal motion segment. For example, the stabilizers may be connected to the spinous process, the transverse process, the lamina, the inferior articular facet, the superior articular process, the pedicle, etc.

At least one portion of the stabilizer may also be connected, attached, secured, fitted, etc., (collectively, connected) to a variety of bodies that are not part of the spinal motion segment. For example, the stabilizer may be connected to other boney tissue, and/or to another spinal implant such as a metal rod, hook, screw, nail, or cage.

The body to which the stabilization member is connected or secured, whether part of the spine or not, should be sufficiently stable and strong to allow the stabilization member to cooperate with the other body to stabilize the portion of the spine to which the stabilization member is connected or secured.

As previously indicated, the stabilizer may have elastic properties that enable it to be stretched by flexion and/or extension movement of the spine. In such embodiments the stabilizer may be sized and configured to provide elastic forces to the spine when the stabilizer is stretched, and may thereby restrict the range of motion of the segment when the segment is subjected to flexion or extension movement. The elastic forces further may act to return the spine to its proper alignment before excessive motion can occur.

In some embodiments the bioresorbable and/or biointegrable material used in the invention may be augmented with one or more therapeutic agents to promote healing and/or bone regeneration. Such additives may include, for example, analgesics, antibiotics, proteoglycans, growth factors, bone morphogenic proteins (BMP), stem cells, steroids, anti-inflammatories, and/or other cells effective to promote healing and/or proper spine function. Additionally or alternatively, radiocontrast materials may be included in the tissue to enhance imaging of the implanted stabilizer.

As indicated above, the stabilizers may additionally or alternatively comprise, consist of, or consist essentially of a blocking portion (or several blocking portions) sized and configured to be effective for maintaining a medically desired distance between two adjacent spinous processes when the natural tissue blocking member is positioned between the two spinous processes in a the spine of a medical patient. In general terms the blocking portion blocks the adjacent spinous processes from being compressed to closely together during movement of the spine, and thus avoids allowing the spinous processes to adopt a medically undesired relationship.

The specific distance that is medically desired may vary from case-to-case, but generally it is understood that a spacing of ⅛″ to 1″ is medically desired for many patients. In some patients a spacing of ¼″ to ¾″ between the adjacent spinous processes is medically desired. Since the inventive stabilizers may be used to treat a variety of medial conditions, and since the stabilizers may be used in various parts of the spine, it is understood that a greater or lesser spacing may be medically desired in a particular case.

The blocking portion may be a single, solid block of material, or it may comprise one or more pieces such as sheets which are folded, stacked, or rolled together. Moreover, the blocking portion may be fashioned in any of a variety of shapes, including but not limited to rolls, square or rectangular blocks, cylinders, U-shapes, and laminated layers. Indents effective for facilitating placement between adjacent spinous processes may be included in the blocking portion, regardless of its predominant shape.

When natural tissue is used to make the blocking member of the present invention, the natural tissue may be derived from substantially any natural tissue providing the appropriate mechanical and immunological properties, i.e., any natural tissue capable of providing the correct compliance and implant substance to be integrated into the body and allow for stabilizing spinal motion segments. In some embodiments the natural tissue may comprise and/or be derived from soft tissue such as skin, pericardium, tendon, ligament, fascia, muscle, cartilage, intestine, meniscus, etc. Examples of preferred sources of natural tissue include fascia lata, planar fascia, anterior or posterior cruciate ligaments, patella tendon, hamstring tendons, quadriceps tendons, Achilles tendons, skins, and other connective tissues. The material may be autogenic (autograft), allogenic (allograft), or xenogenic (xenograft), or it may be of human-recombinant origin. The tissue can be of human origin (e.g. for allograft or autograft into human patients) or of non-human origin (e.g. for xenograft implant into human patients).

In some embodiments the material used to make the blocking member is a nonosteogenic natural tissue. The material may be bioresorbable and/or biointegrable, for example, natural tissue. Examples of such nonosteogenic natural tissue include fascia lata, planar fascia, anterior or posterior cruciate ligaments, patella tendon, hamstring tendons, quadriceps tendons, Achilles tendons, skins, and other connective tissues that have not been treated to make them osteogenic.

The tissue used to make the blocking member of certain embodiments may be treated with a cross-linking agent to promote cross-linking of the collagen molecules in the tissue. For example, glutaraldehyde or other protein cross-linking agents may be used to promote covalent or non-covalent crosslinks between collagen molecules. Similarly, agents to inhibit protein denaturization may also be included. Crosslinking agents that would be appropriate for use in the claimed invention are known to persons skilled in the art, and may be selected without undue experimentation.

When natural tissue is used for the blocking member, the tissue may be used in its fresh form, or it may be frozen and/or dehydrated before use. In some embodiments the blocking member is made of natural tissue that has not been processed other than to affect its size or shape.

In other embodiments of the blocking member of the present invention, a bioresorbable and/or biointegrable material other than natural tissue may be used. For example, all or portions of the inventive blocking member may comprise one or more bioresorbable polymers such as poly-L-lactic acid (PLLA), poly-DL-lactic acid (PDLLA), and/or polylactide (PLa, a copolymer of 70:30 Poly(L-lactide-co-D,L-lactide). In some embodiments the synthetic bioresorbable blocking member is nonosteogenic.

It is to be appreciated that in some embodiments at least a portion of the inventive blocking member may be made from materials that are not bioresorbable, including natural or synthetic polymers, rubbers, metals, ceramics, and/or composites.

Regardless of the material used, a blocking member may have elastic properties so that they initially compress when subject to movement of the spine, yet they resist further compression when excessive movement might otherwise occur. A blocking member may thus provide elastic forces to return the spine to its proper alignment before excessive compression can occur.

The nature of the materials employed to form the blocking portion of the spacer should be selected so the formed implants have sufficient load bearing capacity. In preferred embodiments, a compressive modulus of at least about 0.1 Mpa is desired, although compressive strengths in the range of about 1 Mpa to about 20 Mpa are more preferred. Most preferably the compressive modulus is at least about 2 Mpa.

The stabilizers may comprise a stabilizing strap in combination with a blocking member to protect against both excessive extension and excessive compression of the spine. In some embodiments the stabilizing strap additionally serves as a retaining strap effective for holding the blocking portion in position between two, adjacent spinous processes. The retaining strap thus prevents the blocking portion from being dislodged by movement of the spine. In other embodiments the retraining strap may function merely to retain the blocking member, and need not itself provide additional stabilization against vertical or translational instability.

In other embodiments the blocking member may be retained with a separate loop of material that may be threaded through the blocking member and may be looped around one or more boney protrusions, such as the spinal processes. The retaining loops may be made of tendon or another natural material that is effective for holding the natural tissue blocking member in position between two, adjacent spinous processes when the loops are positioned around a spinous process. The loops may be elastic and may loop over adjacent spinous processes with sufficient tension to hold the blocking portion firmly in position.

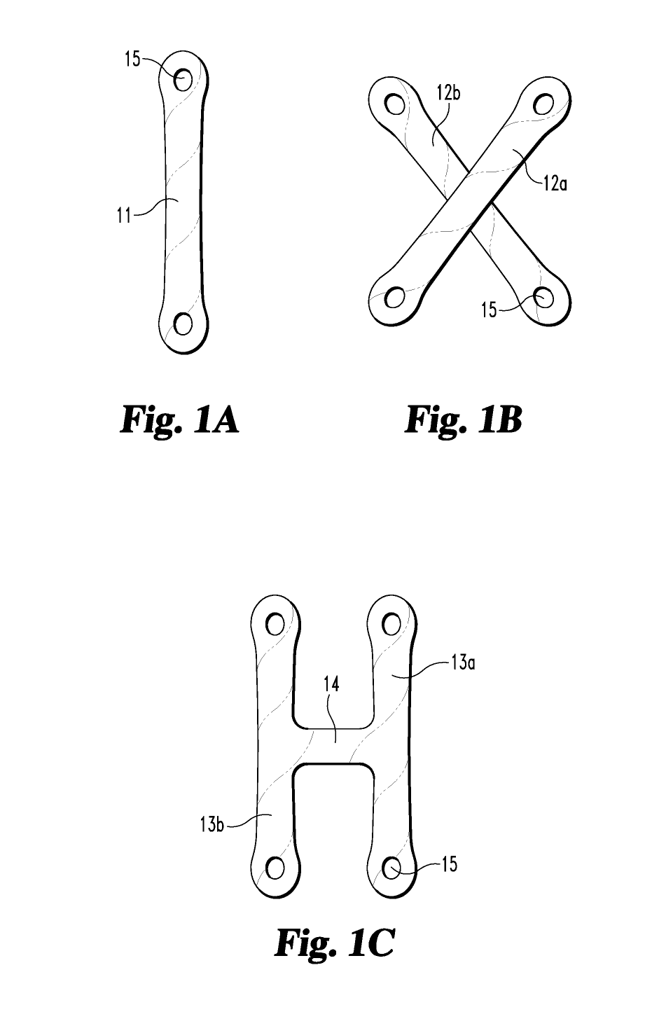

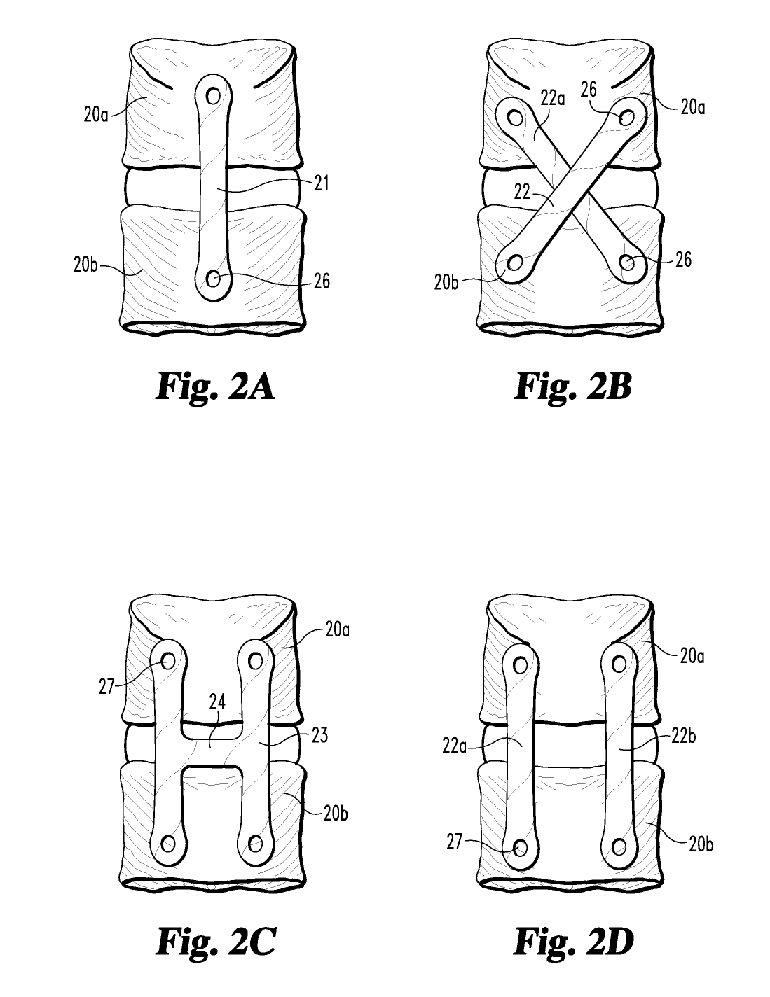

Referring now to the drawings, FIGS. 1A-1C and FIGS. 2A-2D show certain preferred embodiments of the inventive stabilizer. As shown in FIG. 1A, the stabilizer may comprise a single strap 11 of natural tissue, which may be braided. Alternatively, the stabilizer may comprise two or more straps 12, which may be used in combination such as in the “X” configuration formed by straps 12a and 12b illustrated in FIG. 1B. In another alternative, the stabilizer may comprise two or more strap segments 13A and 13B joined by a cross strap 14 to form an “H” shape as illustrated in FIG. 1C. Any or all of straps 11, 12, 13, or 14 may comprise natural tissue in any of its forms, or may comprise another bioresorbable and/or biointegrable material. Other arrangements of one or more straps may be selected without departing from the spirit and scope of the invention.

The stabilizing straps illustrated in FIGS. 1A-1C may be secured to a spine with fasteners such as the bone screws or tacks illustrated herein. The fasteners may be, but need not be, osteogenic. To facilitate the use of such fasteners, the stabilizing straps may be provided with slots or apertures 15 sized to receive said screws or tacks.

FIGS. 2A-2D show stabilizers 21, 22 and 23 positioned to stabilize spinal motion segments. FIG. 2A shows stabilizing strap 21 fastened with bone screws 26 to the lamina or spinal segments 20a and 20b. FIG. 2B shows stabilizing straps 22a and 22b secured to spinal segments 20a and 20b with bone screws 26. FIG. 2C shows an alternate embodiment with “H” shaped stabilizer 23 secured by tacks 27 to stabilize spinal segments 20a and 20b. In an alternative embodiment, a pair of straps 22a and 22b may be used to stabilize spinal segments 20a and 20b by positioning the straps alongside one another, as illustrated in FIG. 2D. The straps may be, but need not be, parallel and/or spaced apart from each other.

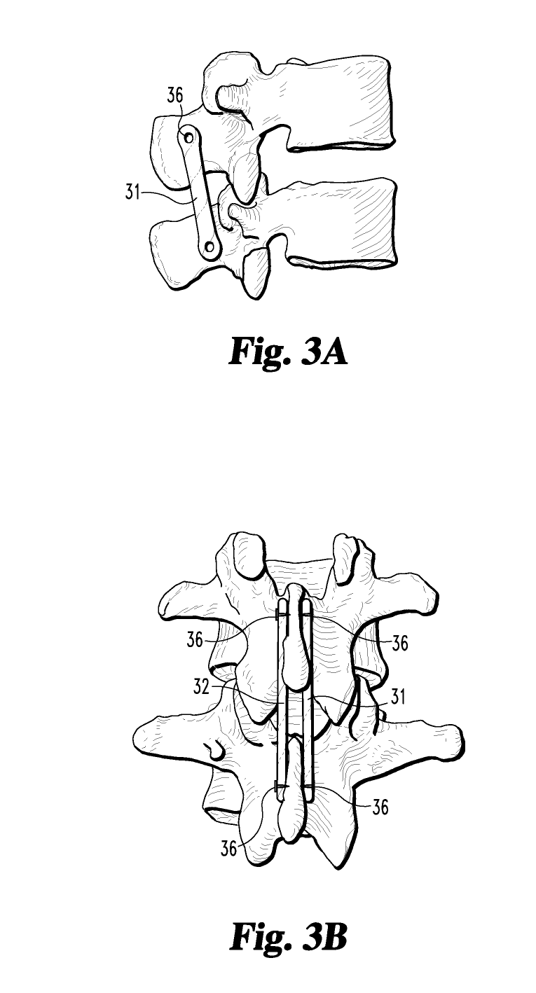

FIGS. 3A and 3B provide alternative views of stabilizing straps secured with screws to a spine. In these views, straps 31 and 32 are secured by screws 36 to opposite sides of the transverse process.

It should be appreciated that the stabilization member may be secured to a spinal motion segment using any type of fastener, including screws, nails, hooks, etc. Such fasteners may be made of a bioresorbable material. The stabilization member may be secured to substantially any boney material, including a vertebral pedicle, lamina, facet, spinous process, transverse process, etc.

It should also be appreciated that any or all of the stabilizing straps may be sized and configured so that it is stretched when it is secured to the spine. In such embodiments, the stabilizer is tensionally secured to the spine, and thus provides elastic forces to reduce the range of motion of the segment(s) to which it is secured.

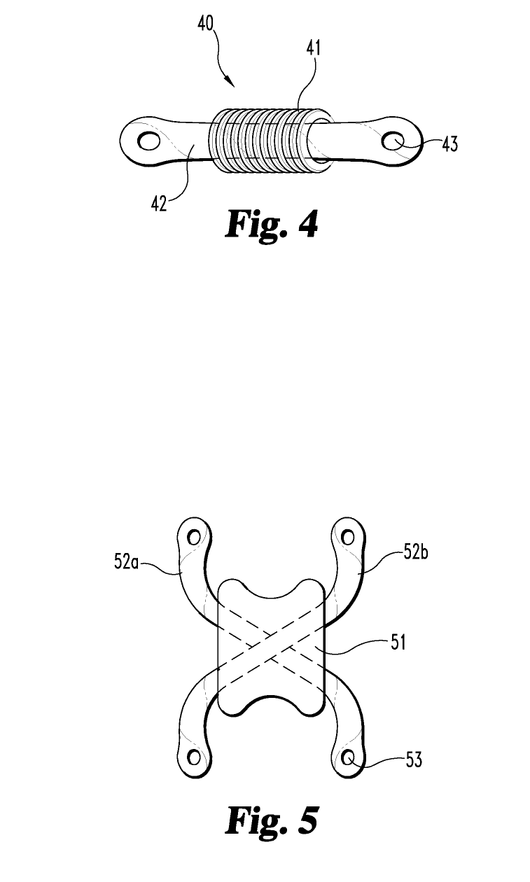

FIGS. 4 and 5 show alternative embodiments in which the stabilizer includes a blocking member. Stabilizer 40 comprises a blocking member 41 and a stabilizing strap 42. Stabilizing strap 42 includes apertures 43 to facilitate securement with screws or pins. Stabilizer 50 includes a blocking member 51 with stabilizing straps 52a, b threaded therethrough. Here too, stabilizing straps 52a, b include apertures 53 to facilitate securement with screws or pins.

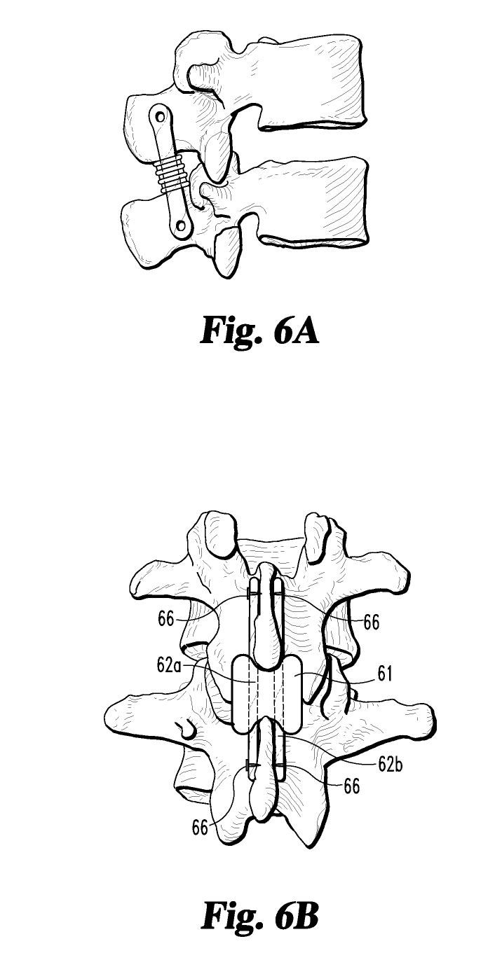

FIGS. 6A and 6B show a stabilizer 60 implanted in a patient. In this embodiment stabilizer 60 includes blocking member 61 secured by straps 62a and 62b and pins 66. Two spinal motion segments are stabilized in the illustrated stabilization, and blocking member 61 ensures that a medically desired distance is maintained between the two adjacent segments.

The stabilizers described above may generally be implanted using an anterior, posterior or lateral approach. The surgeon may utilize a classical “open” surgical technique, or the surgery may be minimally invasive or even percutaneous.

For example, an incision may be made to provide access to the spinal column, and hardware such as pedicle screws may be installed if desired. One portion of the stabilization device may be connected to the spinal motion segment to be stabilized, and another portion of the stabilization member may be connected either to another spinal motion segment to be stabilized, or to another body. In some embodiments the stabilizer may be stretched as it is implanted so that it keeps an elastic force on the spinal motion segment being stabilized. In some embodiments the stabilizer may be implanted so that it stretches with flexion and/or extension movement of the spine, with the elastic forces arising from such stretching pulling the spinal motion segments back together in a manner to prevent excessive flexion and/or extension.

The stabilization devices of the present invention may be used for a variety of therapies, including to stabilize a spine that is being treated by gene therapy, bone morphogenic protein, full or partial nucleus replacement, full or partial disc replacement, or to lesson stenosis, translation or other instability pattern. In one aspect of the present invention, regeneration or cicatrisation of a disc is promoted by stabilizing the spine in conjunction with such therapy.

In one aspect of the present invention the stabilization device is used to stabilize the lumbar region of a spine. Either posterior or anterior stabilization, or both, may be provided.

In a further aspect of the present invention, the extra-discal devices described herein may be used in combination with one or more intra-discal devices that may additionally provide growth factor delivery. For example, intervertebral disc treatment devices and methods as disclosed in U.S. patent application Ser. No. 10/165,347 (Published as U.S. Patent Publication No. US2002/0173851, the contents of which are hereby incorporated herein by reference) may be used in combination with the presently disclosed devices.

While certain aspects of the invention have been described in detail in the drawings and foregoing description, the same are to be considered illustrative of the claimed invention and not limiting, it being understood that all variations and modifications that come within the spirit of the invention are desired to be protected. For example, it is understood that the inventive stabilizers may be used in combination with other spinal instrumentation to provide improved positioning and spacing of two or more segments of a spine.

What is claimed is:

1. A method for stabilizing a spinal motion segment, said method comprising providing a first nonosteogenic bioresorbable stabilization member, securing one portion of said first stabilization member to a spinal motion segment, and securing another portion of said first stabilization member to another body, wherein said securing is effective to restrict the range of motion of the spinal motion segment to which the stabilization member is secured.

2. A method according to claim 1 wherein said first nonosteogenic bioresorbable stabilization member comprises an elongate strap of nonosteogenic, resorbable material.

3. A method according to claim 1 wherein said first nonosteogenic, bioresorbable stabilization member comprises natural tissue material.

4. A method according to claim 1 wherein said first nonosteogenic, bioresorbable stabilization member applies an elastic force to said spinal motion segment, wherein said elastic force is effective to restrict the range of motion of said segment when the stabilization member is stretched by movement of the spine.

5. A method according to claim 1 wherein said first nonosteogenic, bioresorbable stabilization member secures two or more spinal motion segments together to reduce the range of separation between said two or more spinal motion segments.

6. A method according to claim 1 wherein securing one portion of said first stabilization member to a spinal motion segment comprises using bone screws.

7. A method according to claim 1, further comprising providing a second nonosteogenic, bioresorbable stabilization member, securing one portion of said second stabilization member to a spinal motion segment, and securing another portion of said second stabilization member to another body, wherein said securing is effective to restrict the range of motion of the spinal motion segment to which the stabilization member is secured.

8. A method according to claim 7 wherein said second nonosteogenic, bioresorbable stabilization member comprises an elongate strap of resorbable material.

9. A method according to claim 7 wherein said second nonosteogenic, bioresorbable stabilization member comprises natural tissue material.

10. A method according to claim 7 wherein said second nonosteogenic, bioresorbable stabilization member applies an elastic force to said spinal motion segment, wherein said elastic force is effective to restrict the range of motion of said segment when the stabilization member is stretched by movement of the spine.

11. A method according to claim 7 wherein said second nonosteogenic, bioresorbable stabilization member secures two or more spinal motion segments together to reduce the range of separation between said two or more spinal motion segments.

12. A method according to claim wherein securing one portion of said first stabilization member to a spinal motion segment comprises using bone screws.

13. A method according to claim 1, and further comprising:
a) positioning a nonosteogenic blocking member between adjacent spinous processes, said blocking member being sized and configured to be effective for maintaining a medically desired distance between adjacent spinous processes when the natural tissue blocking member is positioned between said spinous processes in a the spine of a patient; and
b) securing said blocking member in position between said spinous processes with at least one retaining member.

14. A method according to claim 13 wherein said blocking member is a bioresorbable blocking member.

15. A device for stabilizing a spinal motion segment, comprising at least one stabilizing member, said stabilizing member comprising nonosteogenic, bioresorbable, and/or bioresorbable material sized and configured to be effective for reducing the range of motion of a spinal motion segment.

16. The device according to claim 15, wherein the nonosteogenic, bioresorbable material comprises an elongate strap.

17. The device according to claim 15, wherein the elongate strap is configured to be effective for reducing the range of motion of a spinal motion segment when one portion of the strap is secured to a spinal motion segment and another portion of the strap is secured to another spinal body.

18. A device according to claim 15 wherein said nonosteogenic, bioresorbable material comprises natural tissue material.

19. A device according to claim 18 wherein said natural tissue comprises one or more members selected from the group consisting of pericardium, fascia, skin, ligament, tendon, intestine, small intestine submucossa, muscle, cartilage, and meniscus.

20. A device according to claim 15 wherein said stabilization member comprises natural tissue treated with a cross-linking solution.

21. A device according to claim 15 wherein at least a portion of said stabilization member is elastic.

22. A device according to claim 15 further comprising a bioresorbable blocking member sized and configured to be effective for maintaining a medically desired distance between adjacent spinous processes when the blocking member is positioned between said two spinous processes in a the spine of a patient.

23. A device according to claim 22 wherein said blocking member comprises natural tissue.

24. A device according to claim 23 wherein said natural tissue comprises tissue derived from one or more members selected from the group consisting of skin, pericardium, tendon, ligament, fascia, muscle, cartilage, intestine, or meniscus.

25. A device according to claim 23 wherein said blocking member is sized to maintain a distance of ⅛″ to 1″ between the spinous processes.

26. A device according to claim 15 comprising at least two straps forming a cross shape or an H-shape.

27. A device according to claim 15 comprising at least one aperture to facilitate attachment of the device to a spinal body.
